(19) United States
(12) Patent Application Publication
(10) Pub. No.: US 2002/0051748 A1
(43) Pub. Date: May 2, 2002

(54) STABILIZED WATER-IN-OIL-IN-WATER ANTIGEN DELIVERY SYSTEM

(76) Inventors: WILLIAM C. SNOW, WINSLOW, ME (US); WALTER C. GOGAN, WINSLOW, ME (US)

Correspondence Address: THOMAS L. BOHAN & ASSOCIATES, 371 FORE STREET, SUITE 202, PORTLAND, ME, 04101, US

(21) Appl. No.: 09/226,525

(22) Filed: Dec. 22, 1998

(51) Int. Cl. A61K 47/44; A61M 36/14; A61K 39/38; A61K 45/00; A61K 47/00; A61K 51/00

(52) U.S. Cl. 424/1.11; 424/204.1; 424/205.1; 424/278.1; 424/283.1; 424/1.29; 424/1.57; 424/1.73

(57) ABSTRACT

A water-in-oil-in-water (W/O/W) emulsion to be used in an antigen delivery system to induce rapid and long-lasting immunity among populations of livestock, birds, and fish. The external aqueous phase of the W/O/W emulsion contains a thixotropic inorganic salt, such as aluminum hydroxide or alum. The presence of the inorganic salt helps to elicit both a Th1 and a Th2 response from the subject's immune system, and the thixotropic properties of the salt stabilize the water-in-oil-in-water emulsion, thereby providing a longer vaccine shelf life. The antigen dose to be delivered to the subject may be contained in entirely in the internal aqueous phase. Alternatively, a first portion of the total antigen dose may be included in the internal aqueous phase and a second portion is included in the external aqueous phase. The incorporation of a portion of the antigen in the external aqueous phase triggers a more uniform immune response across a vaccinated population. The W/O/W based vaccines can be administered either by injection or orally.

### BACKGROUND OF THE INVENTION

1. Field of Invention

The present invention is related to the inducement of rapid and long lasting immunity in vaccinated subjects. More particularly, the invention is related to the parenteral delivery of antigens for inducing immunity in livestock, poultry, and fish. Even more particularly, the present invention relates to a composition to be used in such a delivery system. Still more particularly, the present invention relates to a composition describing a water-in-oil-in-water multiple emulsion for use in an antigen delivery system. Most particularly, the present invention is related to the inclusion of a thixotropic mineral salt adjuvant in the external phase of such a multiple emulsion.

2. Description of the Prior Art

Water-in-oil (W/O) emulsions are widely used to inoculate poultry. Such emulsions consist of microscopic, antigen-containing aqueous droplets suspended in an oil phase, collectively known as micelles. Since emulsions tend to separate into two discrete phases on standing for any appreciable length of time, stabilizing agents are often added in order to keep the aqueous phase dispersed in the continuous oil phase. Thomson (U.K. Patent 1,128,325, issued 1968) describes the preparation of W/O vaccines for cattle, sheep, and lambs in which organic emulsifiers, such as oxidized fatty oils or esters of fatty acids and polyhydric alcohols, are used. Woodhour I (U.S. Pat. No. 3,983,228, issued 1974) and Woodhour II (U.S. Pat. No. 4,069,313, issued 1974) describe W/O vaccines that use isomannide monooleate and aluminum monostearate, either singularly or in combination, to stabilize the emulsion. The use of synthetic polymeric resin adjuvants—i.e., delivery agents—combined with a W/O emulsion system is taught by Glass et al. (U.S. Pat. No. 3,919,411, issued 1975). Midler (U.S. Pat. No. 4,073,743, issued 1978) describes W/O emulsions stabilized by a metal salt of a non-hydrated fatty acid, the salts typically being formed from a fatty acid component derived from monobasic acids that contain 12 to 24 carbons and a metal selected from a group that includes, among others, aluminum, magnesium, iron, and zinc. Aluminum monostearate, having the chemical formula C₁₈H₃₇O₄Al, is the salt used in the preferred embodiment of the vaccine disclosed in Midler. The use of proteins-preferably of human or animal origin-as stabilizing agents for W/O emulsion vaccines was disclosed by Audibert (U.S. Pat. No. 4,125,603, issued 1978).

Due to their well-known “depot effect”, water-in-oil emulsions release antigens slowly. This slow release is beneficial in stimulating a good duration of immunity, but detrimental in building immunity rapidly. Furthermore, oil-based vaccines stimulate an incomplete immune response from the host immune system; a Th1 (cell-mediated) immune response is elicited, but a minimal, if any, Th2 (humoral) immune response is produced as reported by Edelman (Rev. Infect. Dis. 2 (1980) 370) and Bomford (Immunological Adjuvants and Vaccines (Gregoriadis, Allison, and Poste, Eds.) Plenum Press, New York, 1989). Water/oil delivery systems also suffer from compatibility problems with killed bacteria antigens that can lead to adverse reactions following vaccination. Such reactions are typically manifested as lesions around the vaccination site, and can adversely affect egg production, appetite, and feed conversion. In extreme cases, the poultry carcass is ‘condemned,’ i.e., not approved for human consumption.

Another approach to antigen delivery is the use of a mineral salt adjuvant, such as aluminum hydroxide, aluminum phosphate, or calcium phosphate as the delivery agent. These adjuvants act as a binder, carrier, or suspending vehicle for an immunogen or other medicinal agent while stimulating the immune system as well. Once injected, viral or bacterial antigens that are bound to these adjuvants are more rapidly released into the host system, resulting in a faster immune response than that obtained with a corresponding W/O emulsion. However, the duration of immunity obtained with mineral salt adjuvant delivery systems is significantly shorter than with oil emulsions. Aluminum hydroxide is also thixotropic, meaning that it normally has a gelatinous structure, but forms a low viscosity suspension upon the application of a shear force (such as by shaking the container). Vaccines prepared from aluminum hydroxide Al(OH)₃ gel adjuvant, either alone or in conjunction with W/O emulsions, have been used by several investigators, including Blackall et al. (Avian Diseases 31 (1986) 59, Avian Diseases 31 (1987) 527, Avian Diseases 36 (1992) 632) and Matsumoto and Yamamoto (Avian Diseases 15 (1971) 109).

Mineral salt adjuvant delivery systems are more easily injected than more viscous W/O emulsions. Whereas W/O emulsions trigger a Th1 response from the host immune system, adjuvants such as aluminum hydroxide elicit a Th2 response.

A third type of antigen delivery system is the water-in-oil-in-water (W/O/W) emulsion, a type of multiple emulsion. In a W/O/W delivery system, an aqueous phase is dispersed in an oil phase, resulting in the formation of micelles; i.e., a stable suspension of small droplets of i water within a larger oil drop. The water-in-oil micelles are then suspended in a continuous, or external, aqueous phase. W/O/W emulsions have been taught for various applications, including use as vaccine adjuvants, by Hunter et al. (U.S. Pat. No. 5,622,649, issued 1997). Oral vaccination by multiple-emulsions have been reported by Martin et al. (European J. Immunology 27 (1997) 2726) and Tomasi et al. (European J. Immunology 27 (1997) 2720). The W/O/W delivery systems are less viscous than W/O emulsions and are therefore more easily injected and produce fewer vaccination site lesions than water-in-oil delivery systems. Aitken (Immunology 25 (1973) 957) has reported that a W/O/W emulsion is a more effective delivery system than Al(OH)₃ gel adjuvant. Moreover, W/O/W emulsions are capable of providing a longer duration of immunity.

However, multiple-emulsions are physically unstable, as the buoyancy of the oil-containing micelles causes these structures to “cream” to the surface of the continuous aqueous phase. The micelles then coalesce and separation of the aqueous and oil components eventually occurs, causing total failure of the vaccine. One approach to prevent creaming is to add viscosifying agents such as carageenans, guar, locust bean gum, or other synthetic or natural polymers to the external aqueous phase. Although such additives do improve the stability of W/O/W emulsions, they tend to decrease the syringibility and injectability of the emulsion. Like W/O emulsions, W/O/W emulsions stimulate only the Th1 response in immune systems.

Presently, no single parenteral vaccine delivery system: (1) is easily injectable; (2) suitably stimulates both Th1 and Th2 responses in the immune system of subject animals; (3) does not produce severe vaccination lesions; and (4) remains stable during storage. Therefore, what is needed is a parenteral antigen delivery system that possesses the advantages of water-in-oil-in-water multiple-emulsions and gel adjuvant delivery systems. What is also needed is such a delivery system that stimulates a rapid, suitable immune response in the immune system.

What is further needed is a delivery system that provides a good duration of immunity. What is yet further needed is such a delivery system that elicits Th1 and Th2 responses from the immune system, while not producing severe vaccination site lesions. Still further, what is needed is such a physically stable antigen delivery system that will not “cream” and lead to complete separation of the aqueous and oil phases.

### SUMMARY OF THE INVENTION

It is an object of this invention to provide an antigen delivery system that possesses the advantages of multiple water-in-oil-in-water emulsions and gel adjuvant delivery systems. It is also an object of the present invention to provide a parenteral vaccine delivery system that stimulates a rapid, suitable immune response in the immune system. It is a further object of the invention to provide such a delivery system that provides a good duration of immunity. It is yet another object of the present invention to provide such a vaccine delivery system that elicits both Th1 and Th2 responses from the immune system while producing only mild, if any, lesions at the vaccination site. Still further, it is an object of the present invention to provide a physically stable antigen delivery system that will not easily “cream” and separate into aqueous and oil phases upon standing.

These and other objectives are achieved in the present invention by an antigen delivery system that combines the best properties of the water-in-oil, gel adjuvant, and water-in-oil-in-water systems. The system described in the present invention is a composition comprising a water-in-oil-in-water emulsion that is stabilized by the addition of thixotropic inorganic salts, such as aluminum hydroxide or alum, to the continuous, or external, aqueous phase of the emulsion. The thixotropic salt gels upon standing, thereby preventing creaming of the emulsion during storage and coalescence of the micelles. Gel formation does not prevent the vaccine from being administered to livestock, however. Upon application of even a mild shear force, thixotropic materials, by their nature, lose virtually all of their viscosity. An vaccine that includes such a thixotropic salt and has gelled can, once shaken, be readily dispensed.

The thixotropic salt tends to elicit a Th2 response, complementing the Th1 response stimulated by the oil adjuvant. The use of the present invention therefore results in a more complete immune response than that obtained when either the gel or emulsion system is used alone. Furthermore, inclusion of a portion of the antigenic content of the vaccine with the gel adjuvant in the external phase produces a greater immune response than formulations where antigen is not present in the external phase of the W/O/W emulsion or in the gel.

An additional-and originally unforeseen-benefit of the present invention is the increase in consistency of the immune response among a population of vaccinated animals when the mineral salt or external phase antigen is used in a W/O/W emulsion. The uniformity of the vaccine or bacterin performance in vivo is expressed in terms of the coefficient of variation (% CV). A large % CV indicates that some vaccinates may be well protected while others may be poorly protected. The W/O/W emulsion of the present invention has been found to yield low % CV values, thus suggesting a higher level uniformity than those achieved without the combination of gel and emulsion.

These and other advantages will become apparent upon review of the following detailed description of the invention and the appended claims.

### DETAILED DESCRIPTION OF THE PREFERRED EMBODIMENT OF THE INVENTION

Aluminum hydroxide (Al(OH)₃) is the thixotropic salt used in the Preferred Embodiment. In the vaccines of the Preferred Embodiment, the internal aqueous phase (IAP) contains the desired amount of antigen, phosphate-buffered saline (PBS) solution, polyoxyethylene 20 sorbitan monooleate—alternately known as Polysorbate 80—a non-ionic surfactant, and sodium ethylmercurithiosalicylate, a preservative that is also commonly known as thimerosal. The non-ionic surfactant used in the preparation of the vaccines of the Preferred Embodiment is sold under the trade name Tween 80. The oil phase of the Preferred Embodiment is comprised of a light mineral oil, sold under the trade name Drakeol 5, and sorbitan sesquioleate, a non-ionic emulsifier that is sold under the trade name Arlacel 83. The external aqueous phase (EAP) of the Preferred Embodiment contains the desired amounts of aluminum hydroxide (Al(OH)₃), antigen, PBS and the non-ionic surfactant polyoxyethylene 20 sorbitan monooleate (Tween 80). The oil phase, IAP, and EAP for each vaccine of the Preferred Embodiment are premixed individually. The internal aqueous phase and oil phase are then blended together to form a water-in-oil (W/O) emulsion. A water-in-oil-in-water (W/O/W) emulsion is then formed by blending the EAP with the W/O emulsion together at high speed.

The Preferred Embodiment of the present invention is best described by means of examples that serve to illustrate the improved stability and effectiveness of the invention over is the prior art.

### EXAMPLE 1

Four water-in-oil-in water (W/O/W) vaccines were prepared to demonstrate the effectiveness of the present invention in immunizing poultry. Each vaccine contained 1.0×10⁵ EID₅₀/0.5 ml dose of Infectious Bursal Disease Virus (IBDV), standard strain, bursal tissue origin harvest titer of 6.5 EID₅₀/ml, where EID is the egg infection dose required to cause infection in 50% of the eggs. The same equipment, materials, and processing method were used to produce all four vaccines used in the example described herein. All materials and supplies were sterile, and aseptic technique was used throughout the preparation process. The formulations of the four vaccines that were prepared for the examples described herein are listed in Tables 1-4. In vaccine #1 (Table 1), the entire dose of the antigen was contained in the internal aqueous phase and the external aqueous phase contained no mineral salt adjuvant. In vaccine #2 (Table 2), the internal aqueous phase contained all of the antigen as well, but aluminum hydroxide was incorporated into the EAP. Aluminum hydroxide was also included in the EAP of vaccine #3 (Table 3), and 10%, or 1.0×10⁴ EID₅₀, of the antigen was admixed with the Al(OH)₃ in the external aqueous phase, with the internal aqueous phase containing the remainder of the antigen, or 9.0×10⁴ EID₅₀. Vaccine #4 (Table 4) also contained 25% AM(OH)₃ in the EAP, but antigen content was split into two portions: 75% in the IAP and 25% in the EAP.

TABLE 1
Vaccine #1

| IAP | | Oil Phase | | EAP | |
|---|---|---|---|---|---|
| IBDV | 75.95 ml | light | 276.00 ml | PBS | 237.50 ml |
| | | mineral oil | | | |
| 40% Tween ® 80 | 9.00 ml | Arlacel ® 83 | 24.00 ml | 40% Tween ® 80 | 12.50 ml |
| PBS | 214.57 ml | | | W/O emulsion | 250.00 ml |
| | | | | (IAP + oil phase) | |
| 10% Thimerosal | 0.48 ml | | | | |
| | | Total | 600 ml | Total volume: | 500 ml |
| | | volume of | | | |
| | | internal | | | |
| | | aqueous | | | |
| | | and oily | | | |
| | | phases | | | |

TABLE 2
Vaccine #2

| IAP | | Oil Phase | | EAP | |
|---|---|---|---|---|---|
| IBDV | 75.95 ml | light mineral | 276.00 ml | Al(OH)₃ | 125.00 ml |
| | | oil | | | |
| 40% Tween ® 80 | 9.00 ml | Arlacel ® 83 | 24.00 ml | PBS | 112.50 ml |
| PBS | 214.57 ml | | | 40% Tween ® 80 | 12.50 ml |
| 10% Thimerosal | 0.48 ml | | | W/O emulsion | 250.00 ml |
| | | | | (IAP + oil phase) | |
| | | Total | 600 ml | Total volume: | 500 ml |
| | | volume | | | |
| | | internal | | | |
| | | aqueous and | | | |
| | | oily phases | | | |

TABLE 3
Vaccine #3

| IAP | | Oil Phase | | EAP | |
|---|---|---|---|---|---|
| IBDV | 56.96 ml | light mineral | 230.00 ml | Al(OH)₃ | 125.00 ml |
| | | oil | | | |
| 40% Tween ® 80 | 7.50 ml | Arlacel ® 83 | 20.00 ml | IBDV | 6.33 ml |
| PBS | 185.34 ml | | | PBS | 106.17 ml |
| 10% Thimerosal | 0.20 ml | | | 40% Tween ® 80 | 12.50 ml |
| | | | | W/O emulsion | 250.00 ml |
| | | | | (IAP + oil phase) | |
| | | Total volume | 500 ml | Total volume: | 500 ml |
| | | of internal | | | |
| | | aqueous and | | | |
| | | oily phases | | | |

TABLE 4
Vaccine #4

| IAP | | Oil Phase | | EAP | |
|---|---|---|---|---|---|
| IBDV | 47.68 ml | light mineral | 230.00 ml | Al(OH)₃ | 125.00 ml |
| | | oil | | | |
| 40% Tween ® 80 | 7.50 ml | Arlacel ® 83 | 20.00 ml | IBDV | 15.82 ml |
| PBS | 194.62 ml | | | PBS | 96.68 ml |
| 10% Thimerosal | 0.20 ml | | | 40% Tween ® 80 | 12.50 ml |
| | | | | W/O emulsion | 250.00 ml |
| | | | | (IAP + oil phase) | |
| | | Total volume | 500 ml | Total volume: | 500 ml |
| | | of internal | | | |
| | | aqueous and | | | |
| | | oily phases | | | |

Once the vaccines were aseptically produced, sample vials of the different vaccine formulations were tracked at three different temperatures: 4° C.; room temperature (approximately 20° C.); and 37° C. The stability of each of the vaccines was characterized over a period of time by observing the amount of creaming (% C), the amount of separation (% S), and any breakup of the emulsion. These factors were tracked, beginning with the 13th day of storage and ending 100 days later, i.e., on the 113th day of storage. The results of these studies summarized in Tables 5, 6, and 7.

TABLE 5
Vaccine Stability at 4° C.

| Days on | W/O/W control | W/O/W + Al(OH)₃ | | |
|---|---|---|---|---|
| Stability | Vaccine #1 | Vaccine #2 | Vaccine #3 | Vaccine #4 |
| 13 | 5% C* | 15% C | 15% C | 15% C |
| 20 | 5% C | 17.5% C | 17.5% C | 17.5% C |
| 35 | 57.5% C | 20% C | 17.5% C | 17.5% C |
| 44 | 57.5% C | 20% C | 17.5% C | 17.5% C |
| 56 | 57.5% C | 20% C | 17.5% C | 17.5% C |
| 113 | 57.5% C | 17.5% C | 17.5% C | 17.5% C |

*C = amount of vaccine “creaming” observed

TABLE 6
Vaccine Stability at 20° C.

| | | Vaccine #2 | Vaccine #3 | Vaccine #4 |
|---|---|---|---|---|
| Days on | Vaccine #1 | W/O/W + | W/O/W + | W/O/W + |
| Stability | W/O/W control | Al(OH)₃ | Al(OH)₃ | Al(OH)₃ |
| 13 | 5% C* | 15% C | 15% C | 15% C |
| 20 | 5% C | 20% C | 20% C | 20% C |
| 35 | 57.5% C | 22.5% C | 25% C | 22.5% C |
| 44 | 60% C | 25% C | 27.5% C | 25% C |
| 56 | 60% C | 30% C | 15% C | 27.5% C |

| | |
|---|---|
| 113 | data not available for 113 day check |

*C = amount of vaccine “creaming” observed

TABLE 7
Vaccine Stability at 37° C.

| Days on | W/O/W control | W/O/W + Al(OH)₃ | | |
|---|---|---|---|---|
| Stability | Vaccine #1 | Vaccine #2 | Vaccine #3 | Vaccine #4 |
| 13 | 5% C* | 12.5% C | 12.5% C | 12.5% C |
| 20 | 70% C, 17.5% S^{†} | 20% C | 32.5% C | 40% C |
| 35 | 70% C, 25% S | 25% C | 32.5% C, | 45% C |
| | | | 12.% S | |
| 44 | 70% C, 25% S | 25% C | 37.5% C, | 40% C, |
| | | | 17.5% S | 5% S |
| 113 | emulsion broken | 27.5% C | 37.5% C, | 37.5% C, |
| | | | 20% S | 25% C |

*C = amount of vaccine “creaming” observed;
^{†}S = vaccine separation

As seen from the results listed in Tables 5-7, emulsions were less prone to creaming and complete phase separation when the external aqueous phase consisted of 25% Al(OH)_{3.} Compared to vaccine 1, the W/O/W emulsion that did not include Al(OH)_{3,} vaccines 2, 3, and 4 showed more creaming after 13 days and 20 days when stored at 4° C. and 20° C. Thereafter, the creaming rate of the vaccines containing Al(OH)₃ slowed dramatically. In contrast, vaccine 1 continued to cream until the amount of creaming eventually leveled off at a value that was significantly higher than the final amounts of creaming observed in the other three vaccines. In the test conducted at 4° C., vaccines 2, 3, and 4 showed little additional creaming beyond the initial two week period. When stored at room temperature, creaming in the vaccines containing admixed Al(OH)₃ was reduced by 50 to 75% from the value obtained from the control vaccine (vaccine 1). After almost four months at 37° C., the control emulsion of vaccine 1 had completely broken down and was functionally useless. In contrast, vaccines 2, 3, and 4 remained intact over the same period of time at this temperature. These results clearly show that stability of a water-in-oil-in-water emulsion is increased by the addition of mineral salt adjuvants-particularly aluminum hydroxide-to the external aqueous phase of the emulsion.

### EXAMPLE 2

Eighty Specific Pathogen Free (SPF) chickens were taken from the same source and hatch at four weeks of age and split into four groups of 20 chickens each. Individual members of each group were then injected subcutaneously in the neck with one 0.5 ml dose of one of the vaccines listed in Table 8. Levels of infectious bursal disease virus (IBDV) antibodies present in the serum were determined by enzyme linked immunosorbent assay (ELISA) of IBDV antibody titers. The serum was initially collected by bleeding each bird weekly, beginning at one week post-vaccination (WPV) and continuing through 4 WPV. Beyond 4 WPV, the birds were bled biweekly until 14 WPV. After 14 WPV, the birds were re-vaccinated and bled weekly until three weeks post re-vaccination (WPRV). The results of the study are summarized in Table 9.

TABLE 8
Experimental Outline

| | | | Antigen | |
|---|---|---|---|---|
| Group | Vaccine | External Phase | (IAP/EAP) | Birds |
| 1 | W/O/W #1 | PBS | 100%/0% | 20 |
| 2 | W/O/W #2 | Al(OH)₃ | 100%/0% | 20 |
| 3 | W/O/W #3 | Al(OH)₃ | 90%/10% | 20 |
| 4 | W/O/W/ #4 | Al(OH)₃ | 75%/25% | 20 |

TABLE 9
IBDV ELISA titers

| | GMT (% CV) | | | |
|---|---|---|---|---|
| Bleed Schedule | Group 1 | Group 2 | Group 3 | Group 4 |
| 1 WPV | 38ₐ (139.0) | 31ₐ (144.0) | 18ₐ (111.6) | 24ₐ (119.3) |
| 2 WPV | 359ₐ (75.4) | 1001_{b} (39.8) | 1224_{b} (43.2) | 1142_{b} (43.4) |
| 3 WPV | 1608ₐ (47.3) | 2148ₐ (41.3) | 2432ₐ (22.1) | 1731ₐ (35.6) |
| 4 WPV | 2088ₐ (43.4) | 2240_{a,b} (41.7) | 2856_{b} (20.4) | 2558_{a,b} (24.5) |
| 6 WPV | 1956ₐ (51.8) | 2162_{aq,b} (50.5) | 3275_{b} (19.2) | 2778_{a,b} (32.7) |
| 8 WPV | 1578ₐ (56.9) | 1910_{a,b} (49.7) | 2973_{b} (27.4) | 2492_{a,b} (4.06) |
| 10 WPV | 1569ₐ (56.3) | 1937ₐ (50.6) | 2692ₐ (36.5) | 2486ₐ (50.0) |
| 12 WPV | 1868ₐ (58.9) | 1746ₐ (58.6) | 2655ₐ (40.5) | 2550ₐ (46.0) |
| 14 WPV | 1599ₐ (57.6) | 1597ₐ (58.1) | 2173ₐ (47.2) | 2427ₐ (48.5) |
| 1 WPRV | 2102_{a,b} (63.7) | 1634ₐ (72.9) | 2211_{a,b} (59.1) | 3110_{a,b} (56.3) |
| 2 WPRV | 5894ₐ (43.6) | 4832ₐ (44.1) | 7165ₐ (31.6) | 6899ₐ (41.9) |
| 3 WPRV | 5128_{a,b} (41.6) | 4136ₐ (42.9) | 7215_{b} (22.2) | 7005ₐ (30.9) |
| Average % CV | 61.3 | 57.8 | 40.1 | 47.5 |

Note: values within the same row and having the same subscript do not differ significantly from one another. (ANOVA, Duncan test, a = 0.075)

Based on the results listed in Table 9, all three of the vaccines containing aluminum hydroxide had, at two weeks post-vaccination, produced significantly greater IBDV antibody titers than the control (vaccine 1) and thus a faster immune response. Moreover, the two vaccines in which a portion of the antigen was included in the external aqueous phase of the emulsion yielded IBDV antibody titers that were greater than the titers obtained for the vaccines in which the antigen was included only in the internal aqueous phase. This leads to the conclusion that the presence of both aluminum hydroxide and the antigen in the EAP produce the greatest immune response.

The average percent coefficients of variation (% CV) obtained for the four vaccines are also listed in Table 9. A more uniform response by a test group to a vaccine produces a lower value for % CV. As seen in Table 9, vaccines 3 and 4, in which a portion of the antigen was included in the EAP, yielded much lower individual and average % CV values, indicating that incorporation of antigen and aluminum hydroxide in the external phase of a multiple emulsion vaccine triggers a more uniform immune response across the groups tested.

Although the present invention has been described with specific reference to the Preferred Embodiment, it is to be understood that alternative embodiments and equivalents are within the scope of the invention as defined by the appended claims.

I claim:

1. An emulsion for use in vaccines, said emulsion having a total emulsion volume and comprising:
a) an internal aqueous phase;
b) an oil phase; and
c) an external aqueous phase, wherein said continuous external aqueous phase includes at least one thixotropic mineral salt adjuvant.

2. The emulsion as claimed in claim 1 wherein said internal aqueous phase contains at least one component selected from the group consisting of: an aqueous buffer solution; a first emulsifier; a first preservative; adjuvants; live pathogens; killed pathogens; attenuated pathogens; sub-units derived from live pathogens; sub-units derived from killed pathogens; and sub-units derived from attenuated pathogens.

3. The emulsion as claimed in claim 2 wherein said first emulsifier is a non-ionic emulsifier.

4. The emulsion of claim 3 wherein said non-ionic emulsifier is polyoxyethylene 20 sorbitan monooleate.

5. The emulsion as claimed in claim 2 wherein said first preservative is ethylmercurithiosalicylate.

6. The emulsion as claimed in claim 1 wherein said oil phase further comprises an oil and a second emulsifier.

7. The emulsion as claimed in claim 6 wherein said oil is selected from the group consisting of: mineral oils; animal oils; vegetable oils; silicone oils; and vitamin oils.

8. The emulsion as claimed in claim 7 wherein said oil is mineral oil.

9. The emulsion as claimed in claim 6 wherein said second emulsifier is a non-ionic surfactant.

10. The emulsion as claimed in claim 9 wherein said non-ionic surfactant is sorbitan sesquioleate.

11. The emulsion as claimed in claim 1 wherein said vaccine has a total antigen dose, and wherein a first portion of said antigen dose is contained in said internal aqueous phase and a second portion of said antigen dose is contained in said external aqueous phase.

12. The emulsion as claimed in claim 1 wherein said external aqueous phase further contains a water-in-oil emulsion, said water-in-oil emulsion being dispersed throughout said external aqueous phase, wherein said water-in-oil emulsion comprises said internal aqueous phase and said oil phase, and wherein said internal aqueous phase is dispersed within said oil phase.

13. The emulsion as claimed in claim 12 wherein said external aqueous phase further contains at least one component selected from the group consisting of an aqueous buffer solution; a third emulsifier; a second preservative; adjuvants; live pathogens, killed pathogens; attenuated pathogens; sub-units derived from live pathogens; sub-units derived from killed pathogens; and sub-units derived from attenuated pathogens.

14. The emulsion as claimed in claim 13 wherein said third emulsifier is a non-ionic emulsifier.

15. The emulsion as claimed in claim 14 wherein said non-ionic emulsifier is polyoxyethylene 20 sorbitan monooleate.

16. The emulsion as claimed in claim 1 wherein said internal aqueous phase comprises between 5% and 40% of said total emulsion volume, said oil phase comprises between 5% and 75% of said total emulsion volume, and said external aqueous phase comprises between 5% and 95% of said total emulsion volume.

17. The emulsion as claimed in claim 1 wherein said thixotropic mineral salt comprises between about 5% and 95% of said total emulsion volume.

18. The emulsion as claimed in claim 1 wherein said thixotropic mineral salt is selected from the group consisting of aluminum hydroxide and alum.

19. A vaccine for the treatment of livestock, avian species, and fish, said vaccine comprising:
a) a water-in-oil-in-water emulsion, said water-in-oil-in-water emulsion having a total emulsion volume and further comprising an internal aqueous phase, an oil phase, and an external aqueous phase, said external aqueous phase being continuous;
b) one or more antigens; and
c) one or more thixotropic mineral salt, said thixotropic mineral salt being disposed in said external continuous aqueous phase.

20. The vaccine as claimed in claim 19 wherein one of said antigens is contained in said internal aqueous phase.

21. The vaccine as claimed in claim 18 wherein said vaccine has a total antigen dose, and wherein a first portion of said antigen dose is contained in said internal aqueous phase and a second portion of said antigen dose is contained in said external aqueous phase.

22. The vaccine as claimed in claim 21 wherein said first portion is between about 5% and 95% of said total dose and said second portion is between about 5% and 95% of said total dose.

23. The vaccine as claimed in claim 19 wherein said antigens are selected from the group consisting of: live pathogens; killed pathogens; attenuated pathogens; sub-units derived from live pathogens; sub-units derived from killed pathogens; and sub-units derived from attenuated pathogens.

24. The vaccine as claimed in claim 19 wherein one of said antigens is a standard strain of infectious bursal disease virus.

25. The vaccine as claimed in claim 19 wherein said internal aqueous phase further comprises a non-ionic surfactant, a preservative, and a phosphate-buffered saline solution.

26. The vaccine as claimed in claim 25 wherein said non-ionic surfactant is polyoxyethylene 20 sorbitan monooleate.

27. The vaccine as claimed in claim 25 wherein said preservative is sodium ethylmercurithiosalicylate.

28. The vaccine as claimed in claim 18 wherein said oil phase further comprises an oil and a non-ionic emulsifier.

29. The vaccine as claimed in claim 28 wherein said oil is selected from the group consisting of: mineral oil; animal oil; vegetable oil; silicone oil; and vitamin oil.

30. The vaccine as claimed in claim 29 wherein said oil is mineral oil.

31. The vaccine as claimed in claim 28 wherein said non-ionic emulsifier is sorbitan sesquioleate.

32. The vaccine as claimed in claim 19 wherein said external aqueous phase comprises a non-ionic surfactant and a water-in-oil emulsion dispersed throughout said external phase, wherein said water-in-oil emulsion comprises said internal aqueous phase and said oil phase, and wherein said internal aqueous phase is dispersed within said oil phase.

33. The vaccine as claimed in claim 19 wherein each of said thixotropic mineral salts is selected from the group consisting of aluminum hydroxide and alum.

34. The vaccine as claimed in claim 33 wherein said thixotropic mineral salt is aluminum hydroxide.

35. The vaccine as claimed in claim 19 wherein said internal aqueous phase comprises between about 5% and 40% of said total emulsion volume, said oil phase comprises between about 5% and 75% of said total emulsion volume, and said external aqueous phase comprises between about 5% and 95% of said total emulsion volume.

36. The vaccine as claimed in claim 19 wherein each of said thixotropic mineral salts comprises between about 5% and 95% of said total emulsion volume.

37. The vaccine as claimed in claim 19 wherein said vaccine is subcutaneously injectable.

38. The vaccine as claimed in claim 19 wherein said vaccine is administered orally.

39. A method of making a vaccine comprising a water-in-oil-in-water emulsion for treating livestock, avian species, and fish, wherein said water-in-oil-in-water emulsion includes at least one antigen and a thixotropic mineral salt, said method comprising the steps of:
a) preparing an aqueous phase containing a first predetermined dose of an antigen;
b) preparing an oily phase;
c) preparing an emulsion of said internal aqueous phase in said oily phase
d) preparing an external aqueous phase containing a predetermined amount of a mineral salt adjuvant;
e) adding said external aqueous phase to said emulsion, whereby a mixture is formed; and
f) blending said mixture at a predetermined speed for a predetermined time period,
whereby said vaccine is formed.

40. The method of claim 39 wherein the step of preparing said aqueous phase further comprises a step of mixing said predetermined first dose of said antigen, predetermined amounts of a phosphate-buffered saline solution, a non-ionic surfactant, and a preservative together for a predetermined time.

41. The method of claim 39 wherein the step of preparing said oily phase further comprises the step of mixing an oil and a non-ionic emulsifier together for a predetermined time.

42. The method of claim 39 wherein the step of preparing said external aqueous phase further comprises the step of adding a second predetermined dose of said antigen to said external phase.
